(19) United States
(12) Patent Application Publication
(10) Pub. No.: US 2022/0401469 A1
(43) Pub. Date: Dec. 22, 2022

(54) PROCESS FOR PREPARATION OF BIOLOGICALLY ACTIVE COPOLYMER

(71) Applicant: RECCE PHARMACEUTICALS LTD, Western Australia (AU)

(72) Inventors: Graham John Hamilton MELROSE, Western Australia (AU); Michele Keryn DILIZIA, Western Australia (AU); Thomas Gabriel, Wells JARRETT, New South Wales (AU); Arthur KOLLARAS, New South Wales (AU); Justin Mark WARD, Western Australia (AU)

(73) Assignee: RECCE PHARMACEUTICALS LTD, Western Australia (AU)

(21) Appl. No.: 17/871,164

(22) Filed: Jul. 22, 2022

Related U.S. Application Data
(63) Continuation of application No. PCT/AU2021/050063, filed on Jan. 29, 2021.

(30) Foreign Application Priority Data
Jan. 31, 2020 (AU) 2020900270

(51) Int. Cl. A61K 31/765; C08L 71/02; C08G 65/48; A61P 31/04

(52) U.S. Cl. CPC A61K 31/765; C08L 71/02; C08G 65/485; A61P 31/04

(57) ABSTRACT

A process for preparation of a biologically active polymer comprising an acrolein derived segment and a polyalkylene glycol oligomer, the process comprising reacting polyalkylene glycol with acrolein in aqueous solution to form a copolymer of molecular weight no more than 1000 Daltons at a temperature of no more than 15° C.

This is an application filed under 35 USC 111(a), based on PCT/AU2021/050063 filed 29 Jan. 2021, which in turn claims priority to AU 2020900270 filed 31 Jan. 2020. The entirety of the contents of these prior applications are herein incorporated by reference as if set forth herein. All available priority claims to the foregoing applications is made.

### TECHNICAL FIELD

The invention relates to a process for preparation of a biologically active copolymer comprising a segment derived from acrolein monomer and polyalkylene glycol segment. The invention further relates to use of the biologically active agent in treatment of disease, particularly in treatment of bacterial infection, viral infection or cancer.

### BACKGROUND OF INVENTION

There is an increasing need for effective antimicrobial, antiviral and anticancer agents. The evolution of antimicrobial resistance of pathogens has given rise to a serious health risks due to the inability to control infection with many conventional antimicrobials. The emergence of new viral strains leads to a demand for broad spectrum treatments, particularly for serious viral infections. The emergence of the global pandemic during 2020 caused by SARS-CoV-2 has led to millions of infections worldwide a high number of fatalities particularly among the aged and people with comorbidities. This has led to an urgent need for effective treatments of the SARS-CoV-2 infection.

There is also an urgent need for effective treatments of bacterial infection particularly severe and life threatening bacterial infections such as sepsis. The global incidence of and mortality of sepsis in 2017 is estimated at 48.9 million with 11 million sepsis related deaths (The Lancet Vol 395, Issue 10219 pp 200-211 (18 Jan. 2020). Interventions targeting antimicrobial resistance are an imperative to improving sepsis outcomes.

Our co-pending patent applications WO2016/077879 and WO2017/139849 disclose an antimicrobial co-polymer comprising a segment derived from acrolein monomer and polyalkylene glycol segment. The copolymers are formed by reaction of polyacrolein with polyalkylene glycol at a temperature of 25° C. to 35° C. to provide an antimicrobial with good activity against a wide range of bacteria and viruses. The polymeric antibiotics present a significant advancement in treatment of infections such as sepsis due to their activity and reduced propensity of bacterial and viral strains to develop resistance due to the mode of action of the copolymers.

There is an ongoing need to provide biological active compounds of improved activity, particularly antimicrobial activity.

### SUMMARY OF INVENTION

We have now found the biological activity of polymer comprising an acrolein derived segment and a polyalkylene glycol oligomer is significantly increased if the copolymer is formed at low temperature. Accordingly, we provide a process for preparation of a biologically active polymer comprising an acrolein derived segment and a polyalkylene glycol oligomer segment, the process comprising reacting polyalkylene glycol with acrolein in aqueous solution to form a copolymer of molecular weight of no more than 1000 Daltons at a temperature of no more than 15° C., preferably no more than 12° C. such as no more than 10° C. or no more than 8° C.

In a further embodiment there is provided a method of treatment of a subject suffering a disease selected from microbial infection, viral infection and cancer comprising administering to the subject an effective amount of the biologically active agent prepared according to the process.

There is also provided a use of acrolein and polyalkylene glycol in manufacture of a medicament for treatment of a disease selected from bacterial infection, viral infection and cancer wherein the use comprises the process.

The process is particularly suited to treatment of cancer, bacterial infection or viral infection

We have found that the activity of the co-polymer formed at low temperature is very significantly increased. Indeed, the minimum inhibitory concentration (MIC), the lowest concentration which prevents visible growth of a bacterium or bacteria, is very significantly lower at lower temperatures. For example, the MIC is generally at least three-fold higher at 40° C. than at 10° C. Indeed, for many bacteria the MIC is four-fold higher and even at least six-fold higher at 40° C. than at 10° C.

### BRIEF DESCRIPTION OF DRAWINGS

Examples of the invention are described with reference to the accompanying drawings. In the drawings:

FIG. 1 is a graph with four plots referred to in Example 2 comparing the colony-forming unit count (Log₁₀ CFU/swab) of a burn wound infection model treated with (i) the copolymer composition of a process of the invention (E1), (ii) a copolymer composition of comparative process not of the invention (CE3), (iii) a positive control using a known antimicrobial (Saframycin), and (iv) an untreated control.

FIG. 2 is a graph with four plots referred to in Example 2 comparing the wound contraction four days after infection following treatment with the compositions referred to in FIG. 1.

FIG. 3 is a graph with five plots referred to in Example 3 comparing the colony-forming unit count (Log₁₀ CFU/g) of kidney tissue following treatment of ascending urinary tract infection with each of four treatments: the copolymer composition of a process of the invention (E1) at two different treatment rates; a positive control using the antibiotic Meropenem and early and late infection controls.

FIG. 4 is a graph showing five plots referred to in Example 3 comparing the colony-forming unit count (Log₁₀ CFU/g) of bladder tissue following treatment of ascending urinary tract infection with each of four treatments: the copolymer composition of a process of the invention (E1) at two different treatment rates; a positive control using the antibiotic Meropenem and early and late infection controls.

FIG. 5 is a graph showing the nasal wash titres (log 10 genomes/μL) in hamsters infected with SARS-CoV-2 and treated with compositions E1 and CE3 as described in Example 6.

FIG. 6 is a column chart showing the concentration dependent reduction in SARS-CoV-2 virus in an in-vitro study using organoids made from human airway epithelial cells referred to in Example 7.

FIG. 7 is a column chart showing the percent of observed maximum cytotoxicity in a Vero cell protocol of the copolymer of E1 at different concentrations (ppm) referred to in Example 7.

FIG. 8 is a column chart showing the percent of observed maximum cytotoxicity in a Vera cell protocol of the copolymer of CE1 at different concentrations (ppm) referred to in Example 7.

FIG. 9 is a graph showing the bacterial load in vaginal swabs following treatment with references and the compositions of Examples E1 and CE3 referred to in Example 8.

### DETAILED DESCRIPTION

The term “body” means the body of humans and/or animals; the term “subject” means such a body which is the subject.

Intravenous therapy (IV therapy or iv therapy in short) is the infusion of liquid substances directly into a vein.

As used herein, the term “parenteral” means taken into the body in a manner other than through the intact digestive canal. That is, not within the normal stomach or intestine; not intestinal. Parenteral administration of the copolymer prepared by the process is preferred.

The term “parenteral infection” refers to infection contracted by being taken into the body not within the gastro-intestinal tract. Such infection may occur via the vascular (blood/lymph) system, the genital-urinary tracts, from the lungs, disruption of the skin or outer-protective membranes such as in surgery, needle stick injuries, cuts, abrasions, or any break in the skin or gaps between the skin and mucous membranes. It will be understood that a clear distinction is to be made between parenteral infection which may potentially be treated via any method of drug administration including oral administration (assuming an effective dose reaches the site of infection) —and parenteral administration of a drug which is limited to administration other than orally.

As used herein when referring to a bacterial pathogen, the term “antibiotic-resistant” or “superbug” refers to a bacterial pathogen that can withstand an effect of an antibiotic used in the art to treat the bacterial pathogen (i.e., a non-resistant strain of the bacterial pathogen). For example, Staphylococcus aureus can be treated using methicillin; however, an antibiotic-resistant strain of Staphylococcus aureus, S. aureus USA 300, is a methicillin-resistant Staphylococcus aureus (MRSA). Although the bacterial strain is common, S. aureus: USA: 300 typically infects those who are immunocompromised or in a susceptible environment. Infections will often enter the body through a small cut or sore. Other symptoms associated with USA:300 are pneumonia, necrotizing fasciitis, endocarditis, and bone and joint infection.

The term “pulmonary administration” refers to administration of a formulation of the invention into the lungs by inhalation.

The term “polyalkylene glycol” includes linear and branched polyether homopolymers and copolymers of C₂ to C₄ alkylene glycol units. The preferred polyalkylene glycols are polyethylene glycol and polypropylene glycol and copolymers thereof and most preferred is polyethylene glycol.

The term “systemic” refers to a disease or disorder or original site of injury distant to the original site of infection or involving the entire body of the organism. The term “local” therefore is used herein with respect to the site of original infection. Thus, a systemic infection is one in which the pathogen is found in the organs or blood (including bacteremia), and may be associated with a serious, potentially life-threatening disease such as sepsis. A local infection is one in which the pathogen has migrated only as far as the local tissue of infection such as the lung or site of a wound.

As used herein, the term “inhalation” refers to intake of air to the alveoli of the lung. In specific examples, intake can occur by self-administration of a formulation of the invention while inhaling, or by administration via a respirator, e.g., to a patient on a respirator. The term “inhalation” used with respect to a formulation of the invention is synonymous with “pulmonary administration.”

The terms “treatment” and “treating” are intended to encompass also prophylaxis, therapy and cure. Accordingly, in one aspect, a treatment involves preventing or delaying or slowing the onset of a condition, disease, or disorder (e.g. the symptoms associated with the disease, condition, or disorder) associated with antibiotic resistant bacteria. In another aspect, a treatment involves treating (e.g. minimizing or reducing or slowing the development or reversing) an existing condition, disease, or disorder (e.g. the symptoms associated with the disease, condition, or disorder) associated with antibiotic resistant bacteria. In one embodiment, a treatment provides a cure for a condition, disease, or disorder.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject copolymer and/or composition from one organ, or portion of the body, to another organ, or portion of the body. Each carrier must be “acceptable” in the sense of being compatible with the other ingredients of the formulation and not unduly injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The term acrolein-derived segment refers to the copolymer segment comprising one or more acrolein monomer residues.

The terms oligomer, polyalkylene glycol oligomer and polyacrolein oligomer refer to polymers consisting of at least two monomer units, preferably at least three monomer units. The oligomers will typically comprise from 2 to 20 monomer units; in one embodiment the number of units is from 2 to 10.

The terms “monomer units” and “monomer residues” refer to units present in the copolymer derived from the reacting monomers such as acrolein and polyalkylene glycol. Polyalkyleneglycol is considered a prepolymer generally providing at least three glycol monomer units.

The polydispersity index is the ratio of the weight-average molecular weight (M_{w}) of the polymer to the number-average molecular weight (Mₙ) of the polymer. The weight-average molecular weight and the number-average molecular weight of a polymer can be determined by analytical methods, such as high-performance liquid chromatography. Once the weight-average and number-average molecular weights have been determined, the polydispersity index is easily calculated by dividing the weight-average molecular weight by the number average molecular weight, M_{w}/Mₙ. A hypothetically monodisperse polymer has a polydispersity index of 1.000. However, typical commercial polymers, such as the commercially available resins, have a polydispersity index of 10 or more. Polymers with broad molecular weight distributions have higher polydispersity indices and polymers with narrow molecular weight distributions have lower polydispersity indices.

The term “indirect heat exchange” means the bringing of fluids into heat exchange relation without any physical contact or intermixing of the fluids with each other. The cooling fluid is external to the polymerization process.

Throughout this specification, use of the terms “comprises” or “comprising” or grammatical variations thereon shall be taken to specify the presence of stated features, integers, steps or components but does not preclude the presence or addition of one or more other features, integers, steps, components or groups thereof not specifically mentioned.

The process for preparation of the biologically active comprises reacting polyalkylene glycol with acrolein in aqueous solution to form a copolymer of molecular weight of no more than 1000 Daltons at a temperature of no more than 15° C., preferably no more than 12° C., such as no more than 10° C. or no more than 8° C.

Without wishing to be bound by theory it is believed that the formation of more active copolymer with hydrophobic pendant groups is enhanced at low temperature. The reactions between the functional groups within the polymers and the respective target bacteria, virus or cancer are generally enhanced by hydrophobic attractions between them. Hydrophobic attractions, measured in terms of Gibbs Free Energy occur as decreases of enthalpy or increases in Entropy (as equated in the Gibbs thermodynamic Relationship ΔG=ΔH−TΔS) as the result of many inter-actions between especially hydrophobic vinyl or alkylene groups in the polymers, and hydrophobic proteins in the target (bacteria, virus or cancer).

Acrolein monomer (2-propene-one) can react with alkylene glycol oligomers either increasing the chain through the hydrophobic vinyl and leaving the carbonyl pendant, or alternatively, increasing the chain through the carbonyl and leaving the hydrophobic vinyl pendant, and unhindered. The latter of the two alternatives with pendant hydrophobic vinyls, enhance the hydrophobic mechanism.

Without wishing to be bound by theory it is believed that the incidence of polymerization through the carbonyl group of acrolein monomer, rather than through the vinyl group of acrolein monomer, is more significant at relatively low temperatures of no more than 15° C., particularly no more than 12° C. such as no more than 10° C. or no more than 8° C., since the driving-force of the carbonyl polymerization (decrease in enthalpy) is weaker and as a result of de-polymerization at higher temperatures.

Those skilled in the art will be aware that analogues of the present constituent monomers may be synthesized, in alternating or block configurations, to affect hydrophilicity, and hence biological reactivity in manners dictated by their respective monomeric reactivity ratios.

The process typically involves reacting polyalkylene glycol with acrolein in aqueous solution under conditions of alkaline catalysis. Preferably the pH is no more than 12.5 and preferably within a pH range greater than 7.0 to 12.5 such as from 8 to 12.0.

Typically, the aqueous solution of polyalkylene glycol and acrolein comprises water in an amount of at least 20% w/w, such as 20% w/w to 80% w/w, 20% w/w to 70% w/w or 20% w/w to 60% w/w.

The weight ratio of polyalkylene glycol:acrolein is typically at least 4:1 preferably at least 5:1. For example the weight ratio may be about 4:1 to 20:1 such as 5:1 to 20:1 or 10:1 to 15:1

The molecular weight of the copolymer is no more than 1000 Daltons. The copolymer may have a molecular weight of 250 to 1000 Daltons, particularly 300 to 1000 Daltons such as 400 to 1000 Daltons. In the process the polyalkylene glycol is typically of molecular weight of no more than 600 Daltons such as 200 to 600 Daltons.

The process may conveniently be conducted by the addition of acrolein to an aqueous solution of polyalkylene glycol. The aqueous solution of polyethylene glycol may comprise at least 20% w/w water. The addition of polyacrolein to the aqueous polyethylene glycol can be controlled to minimise the effect of the exothermic reaction on increasing the solution temperature. A range of methods known in the art may be used to control the temperature during the reaction of the acrolein monomer and polyethylene glycol. The aqueous solution may be contacted with a heat exchanger to provide cooling of the reaction mixture during the addition of acrolein monomer. For example, temperature may be accurately controlled using a computerized reactor system to maintain the temperature of the aqueous composition based upon starting at the desired temperature.

As the reaction is exothermic, the process preferably includes removing heat of reaction from the zone of reaction of the acrolein monomer and polyalkylene glycol. Heat removal can be achieved by adding additional solvent, such as additional water, or reactants into the reaction zone or by transferring heat from the reaction zone via a commercially available and well-known indirect heat exchange device such as a shell and tube or spiral wound heat exchanger provided with a flow of a cooling fluid such as water.

In one embodiment the effect of the exothermic reaction of acrolein and polyalkylene glycol may be controlled and the temperature maintained in the required range during preparation of the copolymer by carrying out the polymerisation reaction in a reaction vessel provided with indirect heat exchange to maintain the required temperature. In one embodiment the reaction vessel is a jacketed reaction vessel and indirect heat exchange is provided by flow of cooling fluid such as water in the jacket. The process may include maintaining the temperature at no more than 15° C., preferably no more than 12° C. by moderating the flow of cooling fluid and/or the rate of addition of the reactant such as acrolein monomer. The reaction may be conducted in a suitable reactor and commenced at the required temperature of no more than 15° C., preferably no more than 12° C. such as no more than 10° C. or no more than 8° C. and the temperature maintained in the required range.

In a preferred embodiment the heat of polymerisation may be removed from the reactor using indirect exchange with a cooling medium, such as water or other coolant fluid depending on the required temperature, in a jacket surrounding at least part of the reactor. The efficiency of heat removal may be computer controlled to maintain the temperature within the required range during the formation of the copolymer. The reactor may be a batch reactor having a jacket for flow of a cooling fluid, or may be a tubular reactor such as a tubular loop reactor comprising one or more jackets for the cooling fluid, such as water, concentrically surrounding at least part of the tubular reactor.

Typically, the reaction vessel will include an agitator such as a stirrer or other means providing mixing to minimise the occurrence of relatively hot or cold regions as a result of the reaction exotherm and/or heat exchange.

The process may in one set of embodiments be carried out with a temperature in the range of −20° C. to 15° C., preferably no more than 12° C. such as −10° C. to 12° C. or −10° C. to 10° C. Typically the temperature of the reactant solution will be at least −10° C.

In a further set of embodiments, the temperature is in the range of −10° C. to 15° C., such as −5° C. to 15° C. or 0° C. to 15° C. I a further set of embodiments the temperature is in the range of −5° C. to 12° C. or −5° C. to 10° C.

In one set of embodiments the temperature of the aqueous solution is maintained at no more than 10° C., such as no more than 5° C.

The process preferably involves addition of acrolein to an aqueous solution of the polyalkylene glycol. The acrolein may be added to an aqueous solution of polyethylene glycol comprising at least 20% w/w water wherein the acrolein is added in the form of an aqueous solution of acrolein of concentration no more than 50% w/w, preferably no more than 30% w/w acrolein monomer.

The polyalkylene glycol may be a copolymer comprising one of more of ethylene glycol, propylene glycol and butylene glycol monomers which when there are more than one may comprise the monomer units in random or block distribution. The more preferred polyalkylene glycol is polyethylene glycol of molecular weight 200 to 600 Daltons such as 200 to 400 Daltons. It will be understood by those skilled in the art that the term polyethylene glycol preferably does not include diethylene glycol. Polyethylene glycol of average molecular weight 200 to 600 Daltons includes polyethylene glycol of nominal average molecular weight 200 to 600 Daltons wherein the average molecular weight is not more than 110% and not less than 90% (preferably not more than 105% and not less than 95%) of the nominated value. Polyethylene glycol is of formula H—[OCH₂CH₂]ₙ—OH. The average value of n is at least 3 and is generally from 3 to 10 such as 3 to 6 (although the average need not be an integer). Polyethylene glycol is widely available from commercial suppliers in pharmaceutical grades and is sold in specified nominal molecular weights which generally signify that the average molecular weight is not more than 105% and not less than 95% of the nominated value. The viscosities and methods for molecular weight determination are disclosed in USP NF Official Compendium of Standards Volume 11180-1182 [2007 Edition]. In one set of embodiments the polyethylene glycol is of molecular weight from 200 to 400. In some embodiments it may be preferred to use a specific pure oligomer of ethylene glycol such as the compound of formula

H—[OCH₂CH₂]ₙ—OH where n is 3 or 4.

The invention also provides a method of treatment of a subject suffering a disease selected from microbial infection, viral infection and cancer comprising administering to the subject an effective amount of the biologically active agent prepared according to the process.

The invention may also be expressed as use of acrolein and polyalkylene glycol in manufacture of a medicament for treatment of a disease selected from bacterial infection, viral infection and cancer wherein the use comprises the process.

The copolymer prepared by the process is particularly suitable for administration for treatment of parenteral disease selected from cancer, viral infection or bacterial infection.

The copolymer is typically administered systemically, for example, by oral administration, inhalation, transdermal delivery or by injection such as into the blood stream or intramuscular injection or by intravenous therapy such as by injection or infusion. It is generally accepted that molecules of molecular weight no more than about 1000, particularly less than about 800 Daltons have reasonably free passage across the abdominal membranes. Oral administration requires that the copolymer is absorbed through the gut wall and into the systemic circulation. In this embodiment it is particularly preferred that the copolymer administered orally is of molecular weight no more than 1000 Daltons such as a molecular weight in the range of from 250 to 800 Daltons such as 300 to 800 or 350 to 800. We have found that copolymers of this molecular weight, when administered orally, are transported into the systemic circulation to provide treatment of parenteral viral infection. The proportion of the copolymer absorbed through the gut wall is generally greater for copolymers of lower molecular weight in this range.

The copolymer may be applied as an aerosol, gel, topical foam or ointment or impregnated into a dressing for application to skin or mucous membranes for transdermal or transmucosal delivery. The copolymer may be applied as an inhalation via an aerosol or the like.

In a further embodiment the copolymer is administered by transdermal delivery from a composition which may comprise a penetration enhancer for the polymer. Patches, micro-needles or like devices may be used to enhance transdermal delivery.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol, sorbic acid, and the like. It may also be desirable to include agents to adjust tonicity such as sugars, sodium chloride, and the like. Prolonged absorption of the injectable pharmaceutical form may be brought about by the inclusion of agents which delay absorption such as aluminum monostearate and gelatin.

In another preferred embodiment, the pharmaceutical composition is in a form suitable for sub-cutaneous (s.c.) administration.

Pharmaceutical dosage forms suitable for oral administration include tablets (coated or uncoated), capsules (hard or soft shell), caplets, pills, lozenges, syrups, solutions, powders, granules, elixirs and suspensions, sublingual tablets, wafers or patches such as buccal patches.

The copolymer may be formulated in an aqueous composition as it is soluble and remains soluble over the full 1 to 14 range of pH. The copolymer may be administered in compositions with known pharmaceutically-acceptable carriers and excipients; however aqueous formulations provide a significant advantage. The composition may comprise a wide range of concentrations of the copolymer depending on the specific virus to be treated and mode of administration. In one set of embodiments the concentration of the copolymer in an aqueous pharmaceutical composition is in the range of from 0.01% by weight to 20% by weight of the composition. Accordingly, in a preferred set of embodiments the copolymer is administered as an aqueous solution.

The composition may be administered orally in the form of a tablet, caplet, syrup or liquid and the dose administered orally will depend on the severity and type of virus but may be in the range, for example, of from 1 mg to 1000 mg per kilogram of bodyweight per day, such as from 10 mg to 500 mg per kilogram of bodyweight per day.

In a further aspect the invention provides a composition for treatment of a disease in a subject selected from bacterial infection, viral infection and cancer wherein said composition is prepared by a process herein described.

One of the significant advantages of the copolymer and method of treatment is that they may be used against infections from a wide range of pathogens and in particular is useful in treatment of bacterial infections which may rapidly escalate to present a serious threat such as bacteremia or sepsis or pneumonia or meningitis or cellulitis. Specific examples of such bacterial infections may be selected from the group of bacteria consisting of Proteus spp, Serratia spp, Pseudomonas aeruginosa, Neisseria meningitidis, Escherichia coli, Klebsiella pneumoniae, Staphylococcus aureus, coagulase-negative Staphylococcus spp, Streptococcus pyogenes, Streptococcus pneumoniae and Enterococcus spp.

The activity against a wide range of pathogens and particularly a wide range of bacteria allows the copolymer to be used as a first line of treatment in serious or life threatening infections where, for example, the severity of the infection may not allow sufficient time to properly identify the responsible bacteria.

The viral infection may be caused by a range of viruses such as coated viruses (e.g. lipid coated viruses) including herpes, HIV, cytomegalovirus and influenza. Preferably, the viral infection treated and/or controlled by the method of the invention may be HSV-1, HSV-2, Varicella Zoster Virus (in the form of chicken pox or shingles), HCMV, EBV, Herpes 6, Herpes 7, Herpes 8 and SARS-CoV-2.

In another embodiment the virus is influenza virus such as influenza A.

In yet a further embodiment the virus is Ross River virus.

In a further embodiment the virus is a coronavirus including coronaviruses responsible for Severe acute respiratory syndrome.

In one embodiment the viral infection is SARS-CoV-2 also generally referred to as Covid-19.

In a preferred set of embodiments, the process of preparation of copolymers of the present invention comprises the following steps:

providing a mildly basic (preferably of pH no more than 12.5; more preferably of pH 8 to 12.5 such as 9.0 to 12.0) aqueous solution of a polyalkylene glycol (preferably polyethylene glycol of molecular weight in the range of from 200 to 600 Daltons);

mixing the mildly basic solution vigorously to entrain air; adding (preferably slowly over a period such as at least 2 minutes, more preferably at least 5 minutes) acrolein as an aqueous solution of concentration no more than 50% w/w of the acrolein aqueous solution (usually containing preservative);

maintaining the solution at a temperature of no more than 15° C., preferably no more than 12° C. (such as no more than 10° C., or no more than 8° C. during addition of the acrolein to form the copolymer;

and preferably once the acrolein monomer has been consumed, adding acid to provide a pH less than 9 and preferably no more than 8.

Preferably the temperature is controlled to no more than 15 C, preferably no more than 12 C by conducting the reaction in a reaction vessel provided with indirect heat exchanger such as a jacket with a coolant fluid flow (e.g. water flow) about at least part of the reaction vessel.

The molecular weight of the resulting copolymer is controlled by the molecular weight of the polyalkylene glycol, as well as being directly proportional to its hydroxyl concentration. (The polymerization may begin at ambient temperature, then and the exotherm from the polymerization reaction controlled by one or more of the volume of the reaction mixture the amount of solvent such as water, the rate of addition of reactant (such as the rate of addition of acrolein to aqueous polyalkylene glycol) and the use of a reaction vessel with indirect heat exchange.

During the reaction stirring is preferably continued, and the pH maintained mildly basic (preferably of pH no more than 12.5, more preferably of pH 9 to 12.5, 9 to 12 or 9 to 11), only as necessary. The addition of more base and its concentration is minimized so as to lower degradation/side-reactions and to reduce carbonyl or carboxyl formation in the product.

Finally, the pH of the solution may be reduced. In a preferred set of embodiments, the pH is adjusted to near neutral, by the addition of acid. The extremely pungent smell of acrolein is no longer evident in the copolymer product, which is generally formed in at least 99% yield.

The resulting acrolein-copolymers typically have molecular weights in the range of from 250 to 1000 Daltons (such as 300 to 1000 Daltons, 400 to 1000 Daltons or 400 to 800 Daltons). The copolymers are free of turbidity which would be expected from any content of polyacrolein. The weight-ratio of acrolein:polyethylene glycol used in its preparation of the copolymer is preferably between 1:4 and 1:40, and more preferably between 1:8 and 1:20.

The preferred base is an aqueous solution of an alkali hydroxide; more preferably, the alkali hydroxide is sodium hydroxide.

The preferred acid is dilute hydrochloric acid—although acetic acid is useful for pH buffering purposes.

It is preferred that the addition of acrolein to the aqueous solution of polyalkylene glycol takes about 10 minutes—and the reaction to completion, generally takes place about 40 minutes, and preferably is no more than 90 minutes.

Typically, we have found that a reaction time of 50 minutes is suitable to obtain virtually complete conversion to the copolymer product.

The acrolein is preferably added to the aqueous polyalkylene glycol as an aqueous solution—more preferably as a concentration in the range of from 10% to 30% by weight of acrolein monomer, based on the weight of the aqueous acrolein solution to be added to the aqueous polyalkylene glycol solution.

The resulting copolymer has a reactive carbonyl group-content (plus any carboxyl-content) of less than 10%, more preferably less than 5%, and still more preferably zero %.

The acrolein solution usually contains inhibitor, hydroquinone such as no more than 0.5% and typically 0.01 to 0.5% and more preferably 0.1% w/w.

It will be apparent to those in the art that the copolymers herein may be included in a variety of compositions and physical forms. Particularly, compositions and pharmaceutical methods of use, in vivo, will be apparent, taking advantages of slower clearances of the copolymer. Also, it will be apparent that pharmacological advantage may be taken of variance in molecular weight to adjust the rate of penetration through membranes, tissues and organs—and the resultant absorption or distribution within human or animal body; in this context, the lower molecular weight copolymers such as, for example 300 to 800 Daltons are more rapidly absorbed and distributed than copolymers over a molecular weight of 1000 Daltons.

In view of the results herein, it is also conceivable to add protein, particularly broth to enhance in-use antimicrobial activity of the copolymers.

The subject products, herein, are aqueous-soluble and administration to humans/animals may be by the usual methods known in medicine—particularly, by mouth or injection—and are able to be used in any practical pharmaceutical way, alone or in compositions, within organs and tissues, or in contact with or in in vivo vascular systems of either humans or animals. When the copolymers are administered to humans and animals, they can be given per se or as a pharmaceutical composition containing, for example, 0.1% to 99.5% (more preferably, 0.5% to 90%) of active ingredient in combination with a pharmaceutically-acceptable carrier.

It is also known by those in the Art that bacterial infections may lead to cancer, due either to the infection causing chronic inflammation, or the infection releasing cancer-inducing metabolites (Helicobacter pylori leading to cancer is a well-known example). Thus, as the copolymer described within this invention has been found to be practical as an antibiotic drug against bacteria, viruses and cancer activities as described here, should be advantaged and possibly synergistic. Also, as viral infection, particularly by influenza viruses is often associated with bacterial infection as well—again, concurrent anti-viral and anti-biotic properties in the one drug are advantageously synergistic.

The invention will now be described further with reference to the following Examples. It is to be understood that the examples are provided by way of illustration of the invention and that they are in no way limiting to the scope of the invention.

### EXAMPLES

### Example 1 and Comparative Examples—Preparation of Copolymer of MW about 500 Daltons

The following process was conducted in a vessel with a computer-controlled reaction temperature ranging from 10° C. (Composition Example E1 in accordance with the invention) to 40° C. Comparative Example 4 as shown in Table 1.

The reaction was carried out in a glass reactor equipped with a stirrer and water-flow jacket heat exchange and automated computer feedback temperature control during the reaction.

TABLE 1

| Example (E) | Temperature |
|---|---|
| Comparative Example (CE) | (° C.) |
| E1 | 10 |
| CE1 | 20 |
| CE2 | 30 |
| CE3 | 40 |
| CE4 (control) | 40 |

A solution of freshly distilled acrolein (5 g; inhibited with hydroquinone 0.1% w/w) in water (20 g) was slowly added over 10 minutes to a solution of water (20 g) and polyethylene glycol (60 g; MW 200) which had been rendered pH 12 by the addition of 1M aqueous sodium hydroxide; during the 10 minutes, the yellow color of oxidized hydroquinone quickly appeared, then disappeared. During the process the composition was continuously and vigorously stirred to provide copious contact with air. An exothermic and rapid polymerization took-place.

After another 50 minutes, the clear solution was adjusted to pH 7.5 by the addition of 1M aqueous hydrochloric acid; the product was a clear, almost colorless (very pale yellow) solution. All the tests done on the sample and the results herein, were done on a sample without any purification.

The UV-visible, 200-600 nM spectrum of the product only had substantial absorption in the far edge of the 200-300 nM region.

HPLC indicated the polymerization-yield was 99-100% w/w, and any residual acrolein-monomer was less than 1 ppm w/w; when tested down to pH 1 (and up to pH 14), the copolymer remained soluble.

The MIC of S. aureus and E. coli was determined for the copolymer of each of Example 1 and Comparative Examples 1 to 4 and is reported in Table 2 together with the UV Peak detected at different wavelengths shown.

TABLE 2

| | | MIC | MIC | | | |
|---|---|---|---|---|---|---|
| | Temp. | (ppm) | (ppm) | UV ABS | UV ABS | UV ABS |
| Example | (° C.) | S. aureus | E. coli | @230 nm | @265 nm | @272 nm |
| E1 | 10 | 30 | 30 | 0.7779 | 1.9193 | Not |
| | | | | | | detected |
| CE1 | 20 | 30 | 50 | 1.1818 | 1.5537 | Not |
| | | | | | | detected |
| CE2 | 30 | 150 | 150 | 1.1086 | Not | 0.5890 |
| | | | | | detected | |
| CE3 | 40 | 150 | 400 | 1.0808 | Not | 0.7164 |
| | | | | | detected | |
| CE4 | 40 | 150 | 200 | 1.1285 | Not | 0.6262 |
| | | | | | detected | |

### Example 2—Treatment of Burn Infection

This Example examines the efficacy of the compositions of Example 1 and Comparative Example 3 in treating a wound infection molded compound with negative control with no treatment and positive control with saframycin.

Procedure

Experimental Design

TABLE 3

| | | | Total No. |
|---|---|---|---|
| Group | Group ID | Treatment | of animals |
| 1 | Burn wound with infection | No treatment (vehicle, | 5 |
| | (control) | sterile saline, topical, | |
| | | u.i.d, 3 days) | |
| 2 | Burn wound with infection + | (100 μl (19.15 mg/ml), | 5 |
| | Example 1 Composition | topical, u.i.d, 3 days) | |
| 3 | Burn wound with infection + | (100 μl (19.15 mg/ml), | 5 |
| | Comparative Example 3 | topical, u.i.d, 3 days) | |
| | Composition | | |
| 4 | Burn wound with infection + | Safromycin (30 mg, | 5 |
| | Reference Standard | topical, b.i.d, Q = 12 | |
| | | hr, 3 days) | |

Experimental Procedure

Preparing the Inoculum [Day −1]

One day before induction of infection or on day of wound creation, Glycerol Stock Staphylococcus aureus [ATCC 43300] culture was thawed and inoculated into fresh casein soyabean digest (CSD) broth and incubated overnight (preferably inoculation done in the evening) in the shaking incubator (200 rpm) at 37° C.

Burn Wound Creation

The dorsal skin of Wistar male rats of age 6-8 weeks were shaved. Animals were anesthetized by inhalation of 2-3% isoflurane anaesthesia. Depth of anaesthesia was checked by tail pinch. Burn wound was created on the shaved and disinfected surface using molten wax heated up to 80° C. Wax was poured on shaven back of animal through 2 cm×2 cm plastic mold. Wax was allowed to remain on skin till it solidified. Ketoprofen at dose 10 mg/kg was subcutaneously administered to reduce the pain and stress. Animals were housed individually with enrichments.

Induction of Infection

One day after wounding, rats were infected with 100 μl of ˜10⁸ CFU/ml Staphylococcus aureus at the site of skin wound, and the wound site covered with sterile cotton gauze.

Formulation

The vehicle for the copolymer was sterilized 0.9% saline. The copolymer test doses were formulated as: Group 2-383 mg of Example 1 was added to 20 ml vehicle (conc=19.15 mg/ml) and Group 3-383 mg of Comparative Example 3 was added to 20 ml vehicle (conc=19.15 mg/ml).

Treatment

Treatment was started one day post infection. Animals were treated with test and reference compounds for 3 consecutive days as shown in experimental design.

A volume of 100 μl of both Example 1 (conc=19.15 mg/ml) and Example 3 (conc=19.15 mg/ml) were applied to the wound infected area by pipette, followed by spreading the solution uniformly from center towards peripheral end using a sterile bent tip on wound surface and were covered with sterile cotton gauze.

Saframycin (30 mg b.i.d.) was applied to the wound surface with sterile cotton swab.

Observation

Body weight: Body weights were recorded daily through the study.

Clinical signs: Animals were monitored daily for clinical signs through the study period.

Wound Contraction

The changes in wound area were measured, on day 0 and 4, by tracing the wound boundaries on transparent paper. The tracing was then transferred to 1 mm² graph sheet, from which the wound surface area was evaluated. The evaluated surface area was then employed to calculate the percentage of wound contraction by using the following equation:

$$\% wound contraction = \frac{Initial wound size - specific day wound size}{Initial wound size} \times 100$$

Epithelization Period

Period of epithelization was noted as the number of days after wound healing required for the eschar to fall off, leaving no raw wound behind.

Bacteriological Evaluation of the Wound

The whole wound was swabbed with a cotton swab on day 1 and 4 post wounding. The swab was then cut off and placed in a tube containing 1 ml of sterile physiological saline. After mixing with a vortex mixer to release bacterial cells from the swab into the saline, 1000 of undiluted cell suspension or its 100-fold dilutions were plated and incubated on CSD agar plates at 37° C. for overnight (16 h) for bacterial enumeration.

Data Analysis

Body weight, percent wound contraction, and bacterial load for each animal was estimated. Significant differences between group and control means were evaluated using appropriate statistical tests using software Graphpad Prism (v 5.0).

Results

Evaluation of efficacy of the composition of E1 and CE3 in the Burn Wound Infection Model in rats infected with Staphylococcus aureus [ATCC 4330].

TABLE 4
Bacterial Load on skin post treatment

| | Logic CFU/Swab (Mean ± SD) | | |
|---|---|---|---|
| Group | Treatment | Day 1 PI | Day 4 PI |
| 1 | Burn wound with infection | 5.69 ± 0.51 | 5.41 ± 0.44 |
| 2 | Burn wound with infection + | 5.80 ± 0.52 | 4.59 ± 0.24* |
| | Composition E1 | | |
| 3 | Burn wound with infection + | 5.82 ± 0.33 | 5.35 ± 0.71 |
| | Composition CE3 | | |
| 4 | Burn wound with infection + | 5.84 ± 0.35 | 4.87 ± 0.63 |
| | Reference Standard (Safromycin) | | |

*Significantly lower than day 1 (p < 0.05, paired t test)

Summary
Composition E1 Test Dose (100 μl (19.15 mg/ml), topical, 3 days) showed significant reduction in bacterial load on day 4 when compared to day 1, whereas there was no significant reduction in bacterial load in the vehicle control (p>0.05).
CE3 Test Dose (100 μl (19.15 mg/ml), topical, u.i.d, 3 days) showed significantly lower efficacy on day 4 than E1.
Saframycin (30 mg, topical, b.i.d, 0.12 hr, 3 days) did not show significant efficacy on day 4 when compared to day 1 although the mean load was lower.

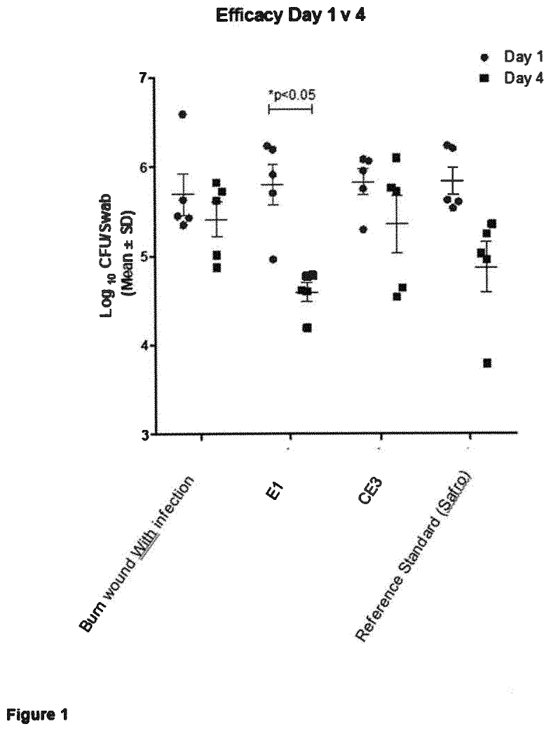

The results are plotted in the graph shown in FIG. 1.

TABLE 5
Percentage Wound Contraction

| | | % wound |
|---|---|---|
| | | contraction |
| | | on Day 4 PI |
| Group | Treatment | (Mean ± SD) |
| 1 | Burn wound with infection | 0.86 ± 0.22 |
| 2 | Burn wound with infection + Composition E1 | 2.56 ± 0.47* |
| 3 | Burn wound with infection + Composition CE3 | 1.95 ± 0.38* |
| 4 | Burn wound with infection + Reference | 2.48 ± 0.30 |
| | Standard (Safromycin) | |

*Significantly different from vehicle control (p < 0.05, 1-way ANOVA)

Summary
Composition E1 Test Dose (100 μl (19.15 mg/ml), topical, u.i.d, 3 days), Composition CE3 Test Dose (100 μl (19.15 mg/ml), topical, 3 days), and Safromycin (30 mg, topical, b.i.d, Q=12 hr, 3 days) showed significant reduction wound on day 4 (p<0.05) when compared to day 1, when compared to the vehicle control.

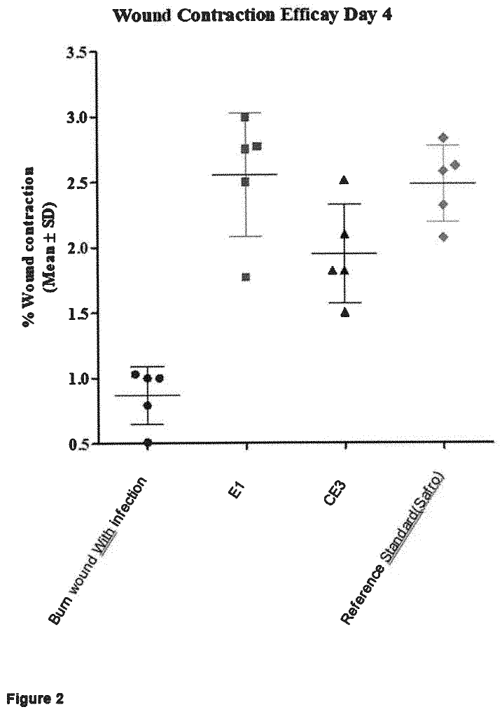

The results are plotted in FIG. 2.

The E1 composition thus exhibited superior in-vivo efficacy compared with CE3, consistent with the lower MIC recorded for the copolymer prepared at temperatures less than 15° C.

### Example 3—Treatment of Ascending Urinary Tract Infection

This Example compares the efficacy of the composition of E1 and CE3 against E. coli in an Ascending Urinary Tract Infection Rat model.

Experimental Procedure

Catheterization of Jugular Vein [2]

Twenty four hours prior to infection, 6-8 week old male Wistar rats were anaesthetized using Ketamine and Xylazine (70+10 mg/kg). Anaesthesia was confirmed by tail pinch. The dorsal and ventral neck surface (3-4 cm) were shaved with a Wahl pet trimmer and surgical site was disinfected with 70% alcohol. The rats were laid in dorsal recumbence and a 1-2 cm incision was made on the ventral neck side; the jugular vein was dissected and made free from surrounding tissues. Two surgical ligatures were placed under the vein (one at cranial end and another at caudal end). The cranial end was knotted tightly, a small incision was given with spring scissors at caudal end, and heparinized saline (100 IU/ml) filled catheter was inserted into the lumen of the vessel (approximately 25-35 mm). Enough blood was withdrawn into the cannula to ensure proper placement and patency.

Preparing the Inoculum [Day −1]

One day before infection (Day −1) a glycerol stock of E. coli [ATCC 25922] was inoculated into sterile Casein Soyabean Digest Broth and kept for incubation at 37° C.

Day 0 (Day of Infection)

On the day of infection, the overnight culture was checked, centrifuged, and the pelleted cells were re-suspended in sterile normal saline and serially diluted to obtain ˜1×10⁹ CFU/ml and used for infection. The inoculum was serially diluted ten-fold in sterile CSD broth and 0.05 ml of six dilutions for each strain was plated over pre-incubated CSD agar plates to determine the viable count (CFU/ml) of inoculum.

Induction of Infection [3,4,5]

Animals with 100% patency were selected for the study and were divided into different groups as specified in the experimental design. Grouped cages of animals were carried to a procedure room, close to a biological safety cabinet. All infections were conducted in a biological safety cabinet, with appropriate personnel protection. Animals were anaesthetized by intraperitoneal injection of Ketamine and Xylazine (60+10 mg/kg i.p.) cocktail. Once the animals were in a sufficiently deep plane of anaesthesia, as monitored by pedal reflex, the abdominal wall of each rat was shaved with electric clippers and the skin was cleansed with 10% povidone iodine. After a 1.5 to 2 cm lower abdominal wall incision, the abdominal wall muscles were separated with blunt dissection. The urinary bladder was isolated and exposed, urine inside the bladder was removed and 0.1 ml of sterile saline or bacterial culture E. coli (˜1×10⁸ CFU/animal) was injected into the bladder. After the replacement of the bladder to its original location, the abdominal muscles were approximated using suture and the skin was closed. The surgical site was cleaned using 10% povidone iodine.

Formulation

The vehicle for Composition E1 was sterilized 0.9% saline. Meropenem was prepared in MilliQ water.

Treatment

Four hours post infection, groups 4, 5 and 6 were administered with Composition E1 intravenously (Jugular vein), as single doses, at a constant rate of infusion (2 ml/kg/hr) over a period of 24 hours in conscious animals, at a dose volume of 48 ml/kg. The dose levels of Composition E1 were 50, 500 and 4000 mg/kg. Group 2 was administered sterilized 0.9% saline intravenously (Jugular vein), as single doses, at a constant rate infusion (2 ml/kg/hr) over a period of 24 hours in conscious animals, at a dose volume of 48 ml/kg. Meropenem (group 3) was administered as a single IV bolus dose of 30 mg/kg (dose volume of 5 ml/kg). The total duration of treatment was 24 hours.

Termination

At 24 hours post initiation of treatment (28 hours post infection), groups of animals were euthanized, as specified in the experimental design, with an overdose of CO₂ in an appropriate exposure chamber. The group 1 animals were sacrificed at 4 hours post infection.

Harvesting Tissue and Homogenization

The euthanized animals were dipped into 70% ethanol for surface decontamination. The organs were removed aseptically—the bladder was cut away near the urethra, and the kidneys were removed by blunt dissection to avoid bleeding. The bladder and each kidney separately were homogenized in sterile saline by using a homogenizer (Omni Tip (THB220 hand held)) and viable counts were determined by using CSD agar plates by spread plate technique. The CFU per milliliter homogenate of bladder and kidney was determined after 18-24 hours of incubation at 37° C.

Data Analysis

The Log₁₀ CFU/gram bladder and Log₁₀ CFU/gram kidneys were estimated in each group. Significant differences between group means were analyzed by one-way ANOVA, followed by a Dunnett's multiple comparison test, using Graphpad Prism at 95% confidence levels. A P value of <0.05 was considered as significant.

TABLE 6
Efficacy of Composition Example E1 against E. coli[ATCC 25922] in an Ascending Urinary Tract infection
Rat Model—Kidneys:

| | | | | | | | | | | | | Mean |
|---|---|---|---|---|---|---|---|---|---|---|---|---|
| | | | | | | | | | | | | Log₁₀CFU/g |
| | | | | | | | | | | | | kidney change |
| | | | | | | | | | | | | (wrt 4 h |
| | | | | | | | | | | | Mean ± SD | PI control: |
| | | | | | | | | | | | (Log₁₀ | 4.73 |
| | Log ₁₀ CFU/g Kidneys | | | | | | | | | | CFU/g | Log₁₀CFU/g |
| Groups | Right kidney | | | | | Left kidney | | | | | kidneys) | kidneys) |
| Early infection | 4.93 | 4.65 | 4.89 | 5.08 | 5.15 | 4.15 | 4.36 | 4.47 | 5.32 | 4.30 | 4.73 ± 0.4 | |
| control | | | | | | | | | | | | |
| Vehicle control | 5.42 | 6.58 | 5.58 | 5.47 | 5.65 | 5.58 | 5.48 | 5.62 | 5.41 | 5.60 | 5.64 ± 0.34 | 0.91 |
| [sterilized 0.9% | | | | | | | | | | | | |
| saline, Single dose, | | | | | | | | | | | | |
| i.v., 24 h infusion] | | | | | | | | | | | | |
| (28 h PI) | | | | | | | | | | | | |
| Meropenem [30 | 3.36 | 3.69 | 3.80 | 3.16 | 3.31 | 3.61 | 3.41 | 3.37 | 3.46 | 3.46 | 3.46 ± 0.19* | −1.27 |
| mg/kg, single dose, | | | | | | | | | | | | |
| i.v., bolus] | | | | | | | | | | | | |
| Composition E1 | 5.43 | 5.36 | 5.77 | 5.10 | 5.35 | 5.58 | 5.43 | 5.45 | 5.30 | 5.58 | 5.44 ± 0.18 | 0.71 |
| [50 mg/kg, single | | | | | | | | | | | | |
| dose, i.v., 24 h | | | | | | | | | | | | |
| infusion] | | | | | | | | | | | | |
| Composition E1 | 4.13 | 4.32 | 4.14 | 4.72 | 4.31 | 5.04 | 5.09 | 4.98 | 4.57 | 4.34 | 4.56 ± 0.37* | −0.17 |
| [500 mg/kg, single | | | | | | | | | | | | |
| dose, i.v., 24 h | | | | | | | | | | | | |
| infusion] | | | | | | | | | | | | |
| Composition E1 | D | D | D | D | D | D | D | D | D | D | NE | NA |
| [4000 mg/kg, single | | | | | | | | | | | | |
| dose, i.v., 24 h | | | | | | | | | | | | |
| infusion] | | | | | | | | | | | | |

*(P < 0.05) Significantly different from vehicle control;
D: Died at 12 h during infusion

Kidneys:
Meropenem showed significant bactericidal activity in kidneys, following a single IV bolus dose of 30 mg/kg when compared to the vehicle control.
Composition E1 [4000 mg/kg] showed toxicity—all animals died at 12 hour post infusion start.
Composition E1 showed dose-dependent antibacterial effect at 50 and 500 mg/kg, with 500 mg/kg showing significant activity when compared to vehicle control (p<0.05).

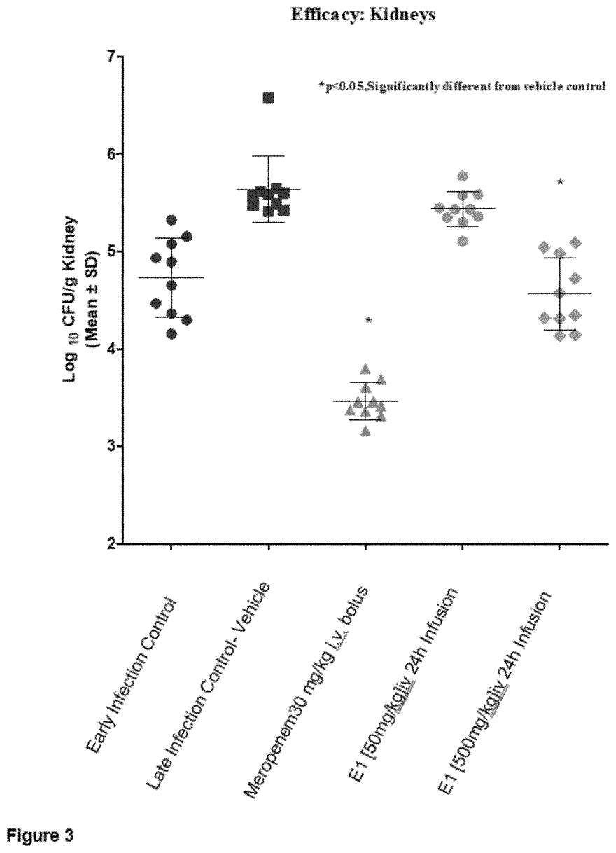

The results are plotted in FIG. 3.

TABLE 7
Efficacy of Composition Example E1 against E. coli[ATCC 25922] in an Ascending
Urinary Tract Infection Rat Model—Bladder Infection Results:

| | | | | | | | Mean Log₁₀CFU/g |
|---|---|---|---|---|---|---|---|
| | | | | | | | Bladder change |
| | | | | | | Mean ± SD | (wrt 4 h PI |
| | | | | | | (Log₁₀ | control: 6.54 |
| | | | | | | CFU/g | Log₁₀CFU/g |
| Groups | Log₁₀ CFU/g Bladder | | | | | Bladder ) | Bladder) |
| Early infection control | 6.00 | 6.41 | 6.63 | 6.84 | 6.80 | 6.54 ± 0.34 | |
| 0.9% saline, Single dose, i.v., | | | | | | | |
| 24 h infusion] (28 h PI) | | | | | | | |
| Vehicle control [sterilized | 9.45 | 8.54 | 8.53 | 8.71 | 9.35 | 8.91 ± 0.45 | 2.37 |
| Meropenem [30 mg/kg, single | 5.04 | 5.24 | 5.91 | 5.25 | 5.71 | 5.43 ± 0.36* | −1.11 |
| dose, i.v., bolus] | | | | | | | |
| Composition E1 | 8.35 | 7.19 | 7.34 | 8.26 | 8.27 | 7.88 ± 0.57* | 1.34 |
| [50 mg/kg, single dose, i.v., | | | | | | | |
| 24 h infusion] | | | | | | | |
| Composition E1 | 6.73 | 7.08 | 6.83 | 5.92 | 6.89 | 6.69 ± 0.45* | −0.15 |
| [500 mg/kg, single dose, i.v., | | | | | | | |
| 24 h infusion] | | | | | | | |
| Composition E1 | D | D | D | D | D | NE | NA |
| [4000 mg/kg, single dose, | | | | | | | |
| i.v., 24 h infusion] | | | | | | | |

*(P < 0.05) significantly different from vehicle control

Bladder
Meropenem showed significant bactericidal activity in the bladder, following a single IV bolus dose of 30 mg/kg when compared to the vehicle control.
Composition E1 showed significant dose-dependent effect in the bladder at 50 and 500 mg/kg (p<0.05) when compared to the vehicle control.

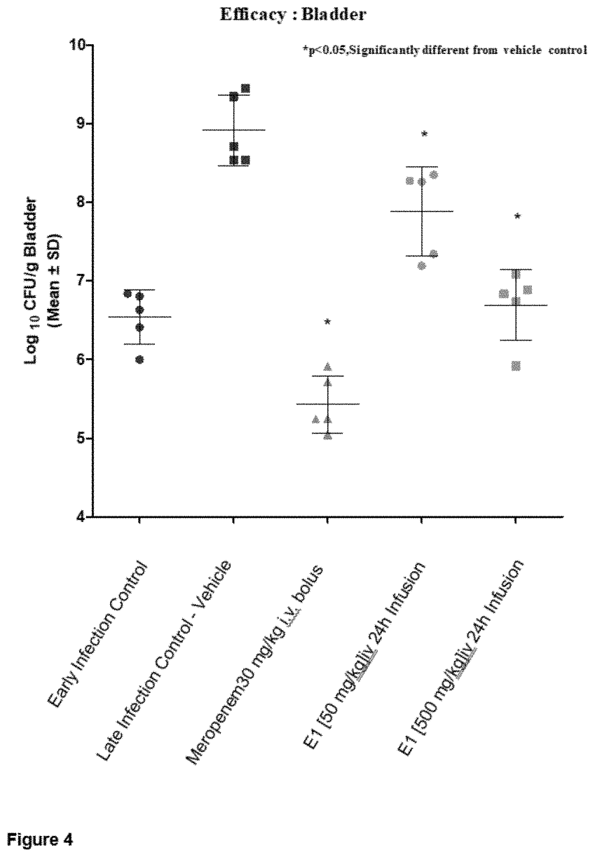

The results of the treatment of bladder infection are shown in FIG. 4.

### Example 4—Influenza A Viral Infection

Summary

The purpose of this study was to determine the efficacy of the composition E1, the composition of CE3 and against Influenza A virus (H1N1) Strain: A/NWS/33 (ATTCC® VR-219™) following intravenous (IV) administration, twice daily for 5 days in C57BL/6 mice. Mice treated with E1 and CE3 showed dose dependent improvement in body weights through the study. Animals in the Ribavarin, E1 (25 mg/kg, 50 and 100 mg/kg) and CE3 (100 mg/kg, 500 and 1000 mg/kg) showed significant higher body weights when compared to the vehicle control (p<0.05) on day 4.

Ribavirin significantly reduced the viral growth rate and viral load in the lungs when compared to the vehicle control; E1 showed significant dose dependent decrease in viral growth rate and viral load in the lungs when compared to the vehicle control. CE3 showed significant dose dependent decrease in viral growth rate and viral load in the lungs when compared to the vehicle control. Animals in the Ribavirin E1 (25 mg/kg, 50 and 100 mg/kg) and CE3 (100 mg/kg, 500 and 1000 mg/kg) showed significantly lower viral titres when compared to the vehicle control (p<0.05) on day 4.

Composition CE3 and Composition E1 in the solutions provided were at concentrations of 1000 mg/ml.

Test System

Species: Mouse

Strain, Sex: C57BL/6, Female

Age: 6-8 weeks

Number of group: 9

Number of animals/group: 12

Total number of animals: 108

Virus: Influenza A virus (H1N1) Strain: A/NWS/33 (ATTCC® VR-219™)

TABLE 8
Experimental Design

| | | Time | Time | Time | Time | |
|---|---|---|---|---|---|---|
| | | Points for | Points for | Points for | Points for | Total |
| | | Lung | Lung | Lung | Lung | No. |
| | | Collection | Collection | Collection | Collection | of |
| Group | Treatment* | PI [Day 1] | PI [Day 2] | PI [Day 4] | PI [Day 6] | Animals |
| 1 | Normal control | 3 | 3 | 3 | 3 | 12 |
| 2 | Infection control [vehicle#] | 3 | 3 | 3 | 3 | 12 |
| | Twice daily (IV bolus, q12 h), | | | | | |
| | 5 days | | | | | |
| 3 | Positive control (Ribavarin, 66 | 3 | 3 | 3 | 3 | 12 |
| | mg/kg, once daily, IP), 5 days | | | | | |
| 4 | E1, 25 mg/kg, | 3 | 3 | 3 | 3 | 12 |
| | Twice daily (IV bolus, q12 h) | | | | | |
| | 5 days | | | | | |
| 5 | E1, 50 mg/kg, | 3 | 3 | 3 | 3 | 12 |
| | Twice daily (IV bolus, q12 h) | | | | | |
| | 5 days | | | | | |
| 6 | E1, 100 mg/kg, | 3 | 3 | 3 | 3 | 12 |
| | Twice daily (IV bolus, q12 h) | | | | | |
| | 5 days | | | | | |
| 7 | CE3, 100 mg/kg, | 3 | 3 | ³ | 3 | 12 |
| | Twice daily (IV bolus, q12 h) | | | | | |
| | 5 days | | | | | |
| 8 | CE3, 500 mg/kg, | 3 | 3 | 3 | 3 | 12 |
| | Twice daily (IV bolus, q12 h) | | | | | |
| | 5 days | | | | | |
| 9 | CE3, 1000 mg/kg, | 3 | 3 | 3 | 3 | 12 |
| | Twice daily (IV bolus, q12 h) | | | | | |
| | 5 days | | | | | |
| | Total | | | | | 108 |

*Begins 4 h before infection

Induction of Infection

Mice were anesthetized intraperitoneally (i.p.) with Ketamine 60 mg/kg IP+ Xylazine 10 mg/kg. Anesthetized mice were infected intranasally by instillation of 30 μl of the virus inoculum (˜1×10⁴) IAV (Plaque Forming Units (PFU)/mouse) in the nostrils (15 μl/nostril).

Formulations

Compositions of E1, CE3 and Ribavirin were formulated in sterile 0.9% saline.

Treatment

Four hours before infection, animals were administered with test and reference items as shown in experimental design. The animals were treated for a duration of six days as shown in the experimental design.

Termination

Three animals from each group were sacrificed on days 1, 2, 4 and 6 post infections with an overdose of CO₂.

Harvesting Tissue and Homogenization

The euthanized animals were dipped into 70% ethanol for surface decontamination. Lungs were aseptically excised and homogenised in a sterile homogenizer in 1 mL of cold infection media. The samples were centrifuged at 300×g for 5-10 min at RT, supernatant was collected in fresh sterile tubes and stores at 4° C. or at −80° C.

Clinical Observations

Animals were monitored for general clinical signs, morbidity and mortality, Body weights were measured at the end of study.

Data Analysis

The Mean±SD Log₁₀ PFU/g lung was estimated in each group. Significant differences between group means and control were analysed by one way ANOVA, followed by a Dunnett's multiple comparison test, using Graphpad Prism at 95% confidence levels. A P value of <0.05 was considered as significant.

Results

On day 6 PI, two out of 3 animals in the vehicle control group were found dead. In the group E1 (25 mg/kg Twice daily (IV bolus, 2 h), 5 days), two out of 3 animals were weak and the third animal was found dead. Animals in all the other groups were apparently normal through the study.

The body weights of animals through the study are shown in Table 9.

TABLE 9
Body weights of mice infected with H1N1 virus through the treatment

| | Uninfected | | | Vehicle | | | Ribavarin 66 | | | E1, 25 mg/kg | | | E1, 50 mg/kg | | |
|---|---|---|---|---|---|---|---|---|---|---|---|---|---|---|---|
| | control | | | control | | | mg/kg i.p. | | | iv bid | | | iv bid | | |
| | Mean | | | Mean | | | Mean | | | Mean | | | Mean | | |
| Days | (g) | SD | N | (g) | SD | N | (g) | SD | N | (g) | SD | N | (g) | SD | N |
| 0 | 24.00 | 0.95 | 12 | 23.67 | 1.07 | 12 | 23.92 | 1.24 | 12 | 24.00 | 0.95 | 12 | 24.25 | 0.62 | 12 |
| 1 | 24.25 | 0.97 | 12 | 23.08 | 1.08 | 12 | 23.50 | 1.24 | 12 | 23.33 | 1.07 | 12 | 23.75 | 0.75 | 12 |
| 2 | 24.67 | 0.87 | 9 | 21.33 | 1.32 | 9 | 24.22 | 0.83 | 9 | 23.11 | 1.36 | 9 | 22.56 | 0.88 | 9 |
| 4 | 25.00 | 1.10 | 6 | 18.83 | 1.33 | 6 | 24.67* | 0.82 | 6 | 21.67* | 0.82 | 6 | 23.17* | 0.75 | 6 |
| 6 | 25.67 | 0.58 | 3 | 19.00 | NA | 1 | 24.70 | 0.58 | 3 | 19.50 | NA | 2 | 23.33 | 0.58 | 3 |
| | | | | E1, 100 mg/kg | | | CE3, 100 mg/kg | | | CE3, 500 mg/kg | | | CE3, 1000 | | |
| | | | | iv bid | | | iv bid | | | iv bid | | | mg/kg iv bid | | |
| | | | | Mean | | | Mean | | | Mean | | | Mean | | |
| | | | Days | (g) | SD | N | (g) | SD | N | (g) | SD | N | (g) | SD | N |
| | | | 0 | 24.08 | 0.79 | 12 | 24.17 | 0.72 | 12 | 24.33 | 0.65 | 12 | 23.92 | 0.90 | 12 |
| | | | 1 | 23.58 | 1.00 | 12 | 23.50 | 0.80 | 12 | 23.67 | 0.49 | 12 | 23.33 | 0.78 | 12 |
| | | | 2 | 23.67 | 1.00 | 9 | 23.33 | 1.00 | 9 | 23.22 | 0.67 | 9 | 23.00 | 0.87 | 9 |
| | | | 4 | 22.67* | 1.03 | 6 | 20.83* | 1.17 | 6 | 23.67* | 0.52 | 6 | 24.00* | 0.89 | 6 |
| | | | 6 | 24.33 | 0.58 | 3 | 22.33 | 0.58 | 3 | 24.67 | 0.58 | 3 | 24.67 | 0.58 | 3 |

*Significantly different from vehicle control (p < 0.05);
NA: Not Applicable

Animals in uninfected control gained weight; Animals in the vehicle control and E1 (25 mg/kg) groups lost weight significantly through the study; Animals in the positive control group showed no significant decrease in body weight. Animals in E1 (50 mg/kg and 100 mg/kg) groups showed drop in mean body weight on day 2 but showed subsequent increase in body weights; Animals in CE3 (100 mg/kg) showed decrease in body weight through the study, whereas 500 mg/kg and 1000 mg/kg showed no significant decrease on day 6. It was not possible to perform a statistical analysis for bodyweights on day 6 as the number of mice in the vehicle control group was one. Instead the statistical analysis was done on day four. Animals in the Ribavarin, E1 (25 mg/kg, 50 and 100 mg/kg) and CE3 (100 mg/kg, 500 and 1000 mg/kg) showed significantly higher body weights when compared to the vehicle control (p<0.05) on day 4.

The viral titers in lungs of mice are shown in Table 10.

TABLE 10
Viral titers in lungs of mice infected with H1N1 virus through the treatment

| | | | | Ribavarin 66 | | | E1, 25 mg/kg | | | E1, 50 mg/kg | | |
|---|---|---|---|---|---|---|---|---|---|---|---|---|
| | Vehicle control | | | mg/kg i.p. | | | iv bid | | | iv bid | | |
| | Mean | | | Mean | | | Mean | | | Mean | | |
| Day | (g) | SD | N | (g) | SD | N | (g) | SD | N | (g) | SD | N |
| 1 | 4.38 | 0.1 | 3 | 4.29 | 0.06 | 3 | 4.29 | 0.08 | 3 | 4.33 | 0.07 | 3 |
| 2 | 4.66 | 0.1 | 3 | 4.31 | 0.08 | 3 | 4.48 | 0.07 | 3 | 4.35 | 0.28 | 3 |
| 4 | 5.37 | 0 | 3 | 4.38* | 0.18 | 3 | 4.98* | 0.03 | 3 | 4.42* | 0.04 | 3 |
| 6 | 5.32 | NA | 1 | 4.16 | 0.17 | 3 | 4.78 | NA | 2 | 4.19 | 0.30 | 3 |
| | E1, 100 mg/kg | | | CE3, 100 mg/kg | | | CE3, 500 mg/kg | | | CE3, 1000 | | |
| | iv bid | | | iv bid | | | iv bid | | | mg/kg iv bid | | |
| | Mean | | | Mean | | | Mean | | | Mean | | |
| Day | (g) | SD | N | (g) | SD | N | (g) | SD | N | (g) | SD | N |
| 1 | 4.35 | 0.09 | 3 | 4.40 | 0.05 | 3 | 4.39 | 0.09 | 3 | 4.39 | 0.1 | 3 |
| 2 | 4.31 | 0.19 | 3 | 4.54 | 0.19 | 3 | 4.35 | 0.03 | 3 | 4.37 | 0.1 | 3 |
| 4 | 4.14* | 0.03 | 3 | 4.60* | 0.18 | 3 | 4.27* | 0.03 | 3 | BLOQ | NA | 3 |
| 6 | 4.07 | 0.06 | 3 | 4.36 | 0.29 | 3 | 4.09 | 0.02 | 3 | BLOQ | NA | 3 |

*Significantly different from vehicle control (p < 0.05);
NA: Not Applicable;
BLOQ: Below Limit of Quantitation

Ribavirin significantly reduced the viral growth rate and viral load in the lungs when compared to the vehicle control; E1 showed significant dose dependent decrease in viral growth rate and viral load in the lungs when compared to the vehicle control. CE3 showed significant dose dependent decrease in viral growth rate and viral load in the lungs when compared to the vehicle control.

It was not possible to perform a statistical analysis for viral titres on day 6 as the number of mice in the vehicle control group was one. Instead the statistical analysis was done on day four. Animals in the Ribavirin, E1 (25 mg/kg, 50 and 100 mg/kg) and CE3 (100 mg/kg, 500 and 1000 mg/kg) showed significantly lower viral titres when compared to the vehicle control (p<0.05) on day 4.

Conclusions

In the mouse influenza model infected with Influenza A virus (H1N1) Strain: A/NWS/33 (ATCC® VR-219™), animals treated with vehicle lost weight significantly; Ribavirin treated mice showed no significant decrease in body weight; mice treated with E1 and CE3 showed dose dependent improvement in body weights through the study. Animals in the Ribavirin, E1 (25 mg/kg, 50 and 100 mg/kg) and CE3 (100 mg/kg, 500 and 1000 mg/kg) showed significantly higher body weights when compared to the vehicle control (p<0.05) on day 4. Ribavirin significantly reduced the viral growth rate and viral load in the lungs when compared to the vehicle control; E1 showed significant dose dependent decrease in viral growth rate and viral load in the lungs when compared to the vehicle control. CE3 showed significant dose dependent decrease in viral growth rate and viral load in the lungs when compared to the vehicle control. Animals in the Ribavirin, E1 (25 mg/kg, 50 and 100 mg/kg) and CE3 (100 mg/kg, 500 and 1000 mg/kg) showed significantly lower viral titres when compared to the vehicle control (p<0.05) on day 4.

### Example 5—Maximum Tolerated Dose

### Example 5A—MTD Following Repeated Intravenous Bolus Dosing for 7 Days in Mice

Female BALB/c mice were dosed with vehicle, CE3 (5, 50 and 500 mg/kg q12 h, 7 days) and E1 (5, 50 and 500 mg/kg q12 h, 7 days) intravenously (bolus). Post dose, animals were observed for general clinical signs. In summary, The MTD of E1 in mice was found to be 500 mg/kg when dosed intravenously twice daily (q=12 h) for a week. The MTD of CE3 in mice was found to be 50 mg/kg when dosed intravenously twice daily (q=12 h) for a week.

Materials and Methods

Test System

Species: Mouse

Strain, Sex: BALB/c, Female

Age: 6 weeks

Number of groups: 7

Number of animals/group: 5

Total number of animals: 35

The compositions of E1 and CE

3 were provided as liquid formulations of conc 1000 mg/ml,

TABLE 11
Experimental Design

| | Test | | No. of |
|---|---|---|---|
| Group | substance | Dose/route/regimen | Animals/group |
| G1 | Vehicle | Water/IV-bolus, q12 h, 7 days | 5 |
| G2 | E1 | 5 mg/kg/IV-bolus, q12 h, 7 days | 5 |
| G3 | | 50 mg/kg/IV-bolus, q12 h, 7 days | 5 |
| G4 | | 500 mg/kg/IV-bolus, q12 h, 7 days | 5 |
| G5 | CE3 | 5 mg/kg/IV-bolus, q12 h, 7 days | 5 |
| G6 | | 50 mg/kg/IV-bolus, q12 h, 7 days | 5 |
| G7 | | 500 mg/kg/IV-bolus, q12 h, 7 days | 5 |

Experimental Procedure

Animals were dosed orally with vehicle and test substances at different doses twice daily (q12 h), for seven days as shown above. Animals were observed for seven days and were compared with vehicle control group for the following parameters:
1. Body weight just before dosing and up to 7 days post dosing
2. Clinical signs (gait, posture, morbidity) before and immediately after dosing up to 7 days.
3. Animals were observed for mortality.

Formulations

0.9% Normal Saline was used as vehicle for E1 and CE3.

CE3 Compositions

5 mg/kg: 2.0 μl of formulation (1000 mg/ml) was diluted to 2 ml with 1.998 ml of 0.9% Normal Saline was added.

50 mg/kg: 20 μl of formulation (1000 mg/ml) was diluted to 2 ml with 1.980 ml of 0.9% Normal Saline.

500 mg/kg: 200 μl of formulation (1000 mg/ml) was diluted to 2 ml with 1.800 ml of 0.9% Normal Saline.

E1 Compositions

5 mg/kg: 2.0 μl of formulation (1000 mg/ml) was diluted to 2 ml with 1.998 ml of 0.9% Normal Saline was added.

50 mg/kg: 20 μl of formulation (1000 mg/ml) was diluted to 2 ml with 1.980 ml of 0.9% Normal Saline.

500 mg/kg: 200 μl of formulation (1000 mg/ml) was diluted to 2 ml with 1.800 ml of 0.9% Normal Saline.

Formulations were prepared freshly daily before each dose.

Dosing

Animals were dosed intravenously (bolus) with CE3 and E1 at the different doses twice daily (q12 h), for seven days as shown above. The dose volume was 5 ml/kg.

Results

Clinical signs of animals, gross pathology and body weights are shown in Tables 12 and 13.

TABLE 12
Clinical observations in mice administered with vehicle and reference compounds

| | | Clinical signs on Days | | | | | | | | | | | | | | |
|---|---|---|---|---|---|---|---|---|---|---|---|---|---|---|---|---|
| | | Day 1 | | Day 2 | | Day 3 | | Day 4 | | Day 5 | | Day 6 | | Day7 | | |
| | Animal | Dose | Dose | Dose | Dose | Dose | Dose | Dose | Dose | Dose | Dose | Dose | Dose | Dose | Dose | |
| Group | No. | 1 | 2 | 3 | 4 | 5 | 6 | 7 | 8 | 9 | 10 | 11 | 12 | 13 | 14 | Remarks |
| Vehicle | 1 | N | N | N | N | N | N | N | N | N | N | N | N | N | N | — |
| (0.9% | 2 | N | N | N | N | N | N | N | N | N | N | N | N | N | N | — |
| Normal | 3 | N | N | N | N | N | N | N | N | N | N | N | N | N | N | — |
| Saline | 4 | N | N | N | N | N | N | N | N | N | N | N | N | N | N | — |
| | 5 | N | N | N | N | N | N | N | N | N | N | N | N | N | N | — |
| CE3, | 6 | N | N | N | N | N | N | N | N | N | N | N | N | N | N | — |
| 5 mg/kg. | 7 | N | N | N | N | N | N | N | N | N | N | N | N | N | N | — |
| IV-bolus, | 8 | N | N | N | N | N | N | N | N | N | N | N | N | N | N | — |
| q 12 h, | 9 | N | N | N | N | N | N | N | N | N | N | N | N | N | N | — |
| 7 days | 10 | N | N | N | N | N | N | N | N | N | N | N | N | N | N | — |
| CE3, | 11 | N | N | N | N | N | N | N | N | N | N | N | N | N | N | — |
| 50 mg/kq, | 12 | N | N | N | N | N | N | N | N | N | N | N | N | N | N | — |
| IV-bolus, | 13 | N | N | N | N | N | N | N | N | N | N | N | N | N | N | — |
| q 12 h, | 14 | N | N | N | N | N | N | N | N | N | N | N | N | N | N | — |
| 7 days | 15 | N | N | N | N | N | N | N | N | N | N | N | N | N | N | — |
| CE3, | 16 | N | N | N | N | N | N | N | N | N | N | N | N | N | N | — |
| 500 | 17 | N | N | N | D | D | D | D | D | D | D | D | D | D | D | Animal found to |
| mg/kg, | | | | | | | | | | | | | | | | be dead before |
| IV-bolus, | | | | | | | | | | | | | | | | 4^{th} dose |
| q 12 h, | 18 | N | N | N | N | N | N | N | N | N | N | D | D | D | D | Animal found to |
| 7 days | | | | | | | | | | | | | | | | be dead before |
| | | | | | | | | | | | | | | | | 10^{th} dose |
| | 19 | N | N | N | D | D | D | D | D | D | D | D | D | D | D | Animal found to |
| | | | | | | | | | | | | | | | | be dead before |
| | | | | | | | | | | | | | | | | 4^{th} dose |
| | 20 | N | N | N | D | D | D | D | D | D | D | D | D | D | D | Animal found to |
| | | | | | | | | | | | | | | | | be dead before |
| | | | | | | | | | | | | | | | | 4^{th} dose |
| E1, | 21 | N | N | N | N | N | N | N | N | N | N | N | N | N | N | — |
| 5 mg/kg, | 22 | N | N | N | N | N | N | N | N | N | N | N | N | N | N | — |
| IV-bolus, | 23 | N | N | N | N | N | N | N | N | N | N | N | N | N | N | — |
| q12 h, | 24 | N | N | N | N | N | N | N | N | N | N | N | N | N | N | — |
| 7 days | 25 | N | N | N | N | N | N | N | N | N | N | N | N | N | N | — |
| E1, | 26 | N | N | N | N | N | N | N | N | N | N | N | N | N | N | — |
| 50 mg/kg, | 27 | N | N | N | N | N | N | N | N | N | N | N | N | N | N | |
| IV-bolus, | 28 | N | N | N | N | N | N | N | N | N | N | N | N | N | N | |
| q12 h, | 29 | N | N | N | N | N | N | N | N | N | N | N | N | N | N | |
| 7 days | 30 | N | N | N | N | N | N | N | N | N | N | N | N | N | N | |
| E1, 500 | 31 | N | N | N | N | N | N | N | N | N | N | N | N | N | N | |
| mg/kg, | 32 | N | N | N | N | N | N | N | N | N | N | N | N | N | N | |
| IV-bolus, | 33 | N | N | N | N | N | N | N | N | N | N | N | N | N | N | |
| q12 h, | 34 | N | N | N | N | N | N | N | N | N | N | N | N | N | N | |
| 7 days | 35 | N | N | N | N | N | N | N | N | N | N | N | N | N | N | |

N—normal
D—dead

TABLE 13
Body weights of mice administered with vehicle and reference compounds

| | An. | Body weight on days (g)s (Mean ± SD) | | | | | | |
|---|---|---|---|---|---|---|---|---|
| Groups | No. | Day 1 | Day 2 | Day 3 | Day 4 | Day 5 | Day 6 | Day 7 |
| Vehicle (0.9% | 1-5 | 24.40 ± 0.89 | 24.60 ± 0.89 | 25.20 ± 0.84 | 25.60 ± 0.89 | 26.40 ± 0.89 | 26.60 ± 1.14 | 27.20 ± 0.84 |
| Normal Saline) | | | | | | | | |
| CE3, 5 mg/kg, | 6-10 | 25.00 ± 1.22 | 26.00 ± 1.22 | 26.60 ± 1.34 | 26.80 ± 0.84 | 27.00 ± 1.41 | 26.80 ± 1.30 | 26.80 ± 1.10 |
| IV-bolus, q 12 h, | | | | | | | | |
| 7 days | | | | | | | | |
| CE3, 50 mg/kg, | 11-15 | 26.00 ± 1.22 | 26.00 ± 1.00 | 26.60 ± 1.14 | 26.40 ± 1.52 | 26.40 ± 0.89 | 26.60 ± 0.89 | 26.60 ± 0.55 |
| IV-bolus, q 12 h, | | | | | | | | |
| 7 days | | | | | | | | |
| CE3, 500 mg/kg, | 16-20 | 25.20 ± 1.10 | 26.50 ± 0.71 | 25.50 ± 0.71 | 25.50 ± 0.71 | 24.00 ± NE | 25.00 ± NE | 24.00 ± NE |
| IV-bolus, q 12 h, | | | | | | | | |
| 7 days | | | | | | | | |
| E1, 5 mg/kg, | 21-25 | 23.60 ± 1.82 | 24.00 ± 1.58 | 24.40 ± 1.82 | 24.80 ± 2.17 | 25.40 ± 2.07 | 26.20 ± 2.28 | 25.80 ± 2.59 |
| IV-bolus, q 12 h, | | | | | | | | |
| 7 days | | | | | | | | |
| E1, 50 mg/kg, | 26-30 | 22.20 ± 0.84 | 23.20 ± 0.84 | 23.60 ± 0.55 | 23.40 ± 0.55 | 23.80 ± 1.10 | 24.20 ± 1.30 | 24.80 ± 1.30 |
| IV-bolus, q 12 h, | | | | | | | | |
| 7 days | | | | | | | | |
| E1, 500 mg/kg, | 31-35 | 23.60 ± 1.14 | 23.60 ± 0.89 | 23.80 ± 0.84 | 23.80 ± 0.45 | 24.00 ± 0.71 | 24.40 ± 1.14 | 24.60 ± 1.14 |
| IV-bolus, q 12 h, | | | | | | | | |
| 7 days | | | | | | | | |

NE: Not Estimated

Conclusions

Mice treated with low doses of CE3 (5 and 50 mg/kg, i.v., q12 h days, for 7 days) were apparently normal post dose and were comparable to the vehicle groups. In mice dosed with CE3 high dose (500 mg/kg, i.v., q12 h days, for 7 days), three out of five mice were found to be dead post 4th dose.

Animals in the E1 treated groups, i.e., vehicle, low, mid and high dose groups were apparently normal through the study.

Mean body weights increased in both CE3 and E1 treated mice groups, i.e., vehicle, low, mid and high dose groups except CE3 high dose group.

In summary, The MID of E1 in mice was found to be 500 mg/kg when dosed intravenously twice daily (q=12 h) for a week. The MTD of CE3 in mice was found to be 50 mg/kg when dosed intravenously twice daily (q—12 h) for a week.

### Example 5B—Identification of Maximum Tolerated Dose MTD of Compositions of Example CE3 and E1 Following Repeated Oral Dosing for Seven Days in Rat

Summary

Male Sprague Dawley rats were dosed with vehicle, The Composition of CE3 (5, 50 and 500 mg/kg q12 h) and the composition of E1 (5, 50 and 500 mg/kg g12 h) orally for seven days. Post dose, animals were observed for general clinical signs and body weights. The maximum tolerated dose MTD of CE3 and E1, when administered orally, twice daily repeatedly for 7 days, was found to be at least 500 mg/kg in rats.

Testing

The Compositions of CE3 and E1 were supplied as solutions of conc 1000 mg/ml in glass vials.

Test Subjects

Species: Rat

Strain: Sprague Dawley

Age: 6-8 weeks

Sex: Males

Experimental Procedure

| | Test | | No. of |
|---|---|---|---|
| Group | substance | Dose/route/regimen | Animals/group |
| G1 | Vehicle | Water/PO, q12 h, 7 days | 5 |
| G2 | E1 | 5 mg/kg/PO, q12 h, 7 days | 5 |
| G3 | | 50 mg/kg/PO, q12 h, 7 days | 5 |
| G4 | | 500 mg/kg/PO, q12 h, 7 days | 5 |
| G6 | CE3 | 5 mg/kg/PO, q12 h, 7 days | 5 |
| G6 | | 50 mg/kg/PO, q12 h, 7 days | 5 |
| G7 | | 500 mg/kg/PO, q12 h, 7 days | 5 |

Animals were divided into different groups as shown in the experimental design and given feed and water ad libitum. Groups of rats were dosed orally with test substances at the different doses twice daily (q12 h), for seven days as shown above.

Results

Animals dosed with Vehicle, CE3 and E1 at all the doses were apparently normal through the study (first table below 1). The mean body weights of animals increased in all the groups and no apparent abnormalities were observed in gross pathology in all the groups (second table below 2).

| | Clinical signs | | | | | | |
|---|---|---|---|---|---|---|---|
| Group | Days 1-7 | | | | | | |
| Vehicle Water/PO, | NAD | NAD | NAD | NAD | NAD | NAD | NAD |
| q12 h, 7 days | | | | | | | |
| E1 5 mg/kg/PO, q12 | NAD | NAD | NAD | NAD | NAD | NAD | NAD |
| h, 7 days | | | | | | | |
| E1 50 mg/kg, | NAD | NAD | NAD | NAD | NAD | NAD | NAD |
| mg/kg/PO, q12 h, 7 | | | | | | | |
| days | | | | | | | |
| E1 500 mg/kg/PO, | NAD | NAD | NAD | NAD \| NAD | | NAD | NAD |
| q12 h, 7 days | | | | | | | |
| CE3 5 mg/kg/PO, | NAD | NAD | NAD | NAD | NAD | NAD | NAD |
| q12 h, 7 days | | | | | | | |
| CE3 50 mg/kg/PO, | NAD | NAD | NAD | NAD | NAD | NAD | NAD |
| q12 h, 7 days | | | | | | | |
| CE3 500 mg/kg/PO, | NAD | NAD | NAD | NAD | NAD | NAD | NAD |
| q12 h, 7 days | | | | | | | |

NAD = no abnormality detected

| | Group | | | | Body weight(g) (Mean ± SD) | | |
|---|---|---|---|---|---|---|---|
| ss | Day 1 | Day 2 | Day 3 | Day 4 | Day 5 | Day 6 | Day 7 |
| Vehicle Water/PO, | 213 ± 8.09 | 224.4 ± 6.73 | 236.2 ± 4.82 | 246 ± 5.15 | 253.2 ± 4.15 | 262.6 ± 3.65 | 268.2 ± 5.81 |
| q12 h, 7 days | | | | | | | |
| E1, 5 mg/kg/PO, | 214.4 ± 5.03 | 225.6 ± 6.5 | 234 ± 5.96 | 245.2 ± 5.36 | 256.6 ± 9.07 | 262.8 ± 6.38 | 270.2 ± 6.3 |
| q12 h, 7 days | | | | | | | |
| E1, 50 mg/kg, | 212.8 ± 7.56 | 223.2 ± 4.44 | 234 ± 4.24 | 243.4 ± 5.18 | 253.8 ± 5.63 | 263.6 ± 5.22 | 272.4 ± 3.21 |
| 7 days | | | | | | | |
| E1, 500 mg/kg/PO, | | | | | | | |
| q12 h, 7 days | | | | | | | |
| mg/kg/PO, q12 h, | 213.4 ± 4.56 | 223.4 ± 9.32 | 231.6 ± 7.7 | 240 ± 4.74 | 246.8 ± 5.89 | 255.2 ± 9.65 | 269.4 ± 5.77 |
| CE3, 5 mg/kg/PO, | 212.2 ± 8.17 | 223.8 ± 6.8 | 231.8 ± 6.76 | 240.8 ± 5.22 | 249.2 ± 5.07 | 258.2 ± 3.42 | 269 ± 3.32 |
| q12 h, 7 days | | | | | | | |
| CE3, 50 mg/kg/PO, | 216.2 ± 4.21 | 227.2 ± 3.7 | 233.6 ± 3.36 | 241 ± 1.87 | 248.2 ± 3.11 | 257 ± 4.9 | 269.6 ± 4.51 |
| q12 h, 7 days | | | | | | | |
| CE3, 500 | 215 ± 5.96 | 225.4 ± 5.03 | 234.6 ± 4.34 | 243.4 ± 5.55 | 252 ± 5.57 | 259.8 ± 4.71 | 270.4 ± 5.03 |
| mg/kg/PO, q12 h, | | | | | | | |
| 7 days | | | | | | | |

Conclusions

The maximum tolerated dose MTD of CE3 and E1, when administered orally, twice daily repeatedly for 7 days, was found to be at least 500 mg/kg in rats.

### Example 6—SARS-CoV-2

The compositions of E1 and CE2 demonstrated dose-dependent activity in-vivo against SARS-CoV-2 virus in Syrian golden hamsters, a well-accepted model of infection.

Intranasal administration of both compounds supports multiple potential modes of administration against SARS-CoV-2.

Method

The study consisted of five groups of eight hamsters, each receiving a different treatment—placebo control of saline nasal wash, low dose of CE3 (200 mg/kg), high dose of CE3 (400 mg/kg), low dose of E1 (100 mg/kg), and high dose of E1 (200 mg/kg). All animals were infected with SARS CoV-2 on Day 0 with treatments administered twice daily on Days 1-5 and viral titres measured directly on Days 2, 4 and 6 via qPCR. In this model, the viral titres typically peak between Days 2 and 4.

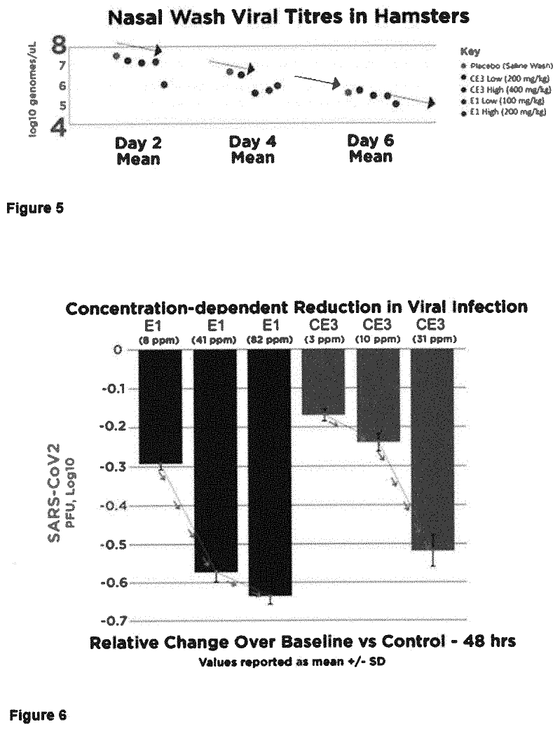

The results, in both CE3 and E1, demonstrated a positive reduction in COVID-19 viral load compared to the placebo group. The results are shown in FIG. 5 of the drawings is a graph showing the nasal wash titres (log 10 genomes/μL) in hamsters infected with SARS-CoV-2 and treated with compositions E1 and CE3. In the graph the plots from left to right for each day relate to placebo, CE3 (200 mg/Kg), CE3 (400 mg/Kg), E1 (100 mg/Kg) and E1 (200 mg/Kg). A mean log reduction within groups on Day 4 where the low E1 dose achieved a log reduction in the order of 1.5 logs and a high dose of CE3 achieved a log reduction of 1.25 logs. Two of the five hamsters with COVID-19 infection on Day 6 indicated adverse clinical symptoms in the high dose E1 group and were excluded from the study. The Company considers a study specific anomaly since E1 was routinely well tolerated at considerably higher intravascularly infused doses in-vivo. The weight of the hamsters across all groups at the start and the end of the study remained approximately the same.

This hamster study demonstrates the potential for nasal administration of E1 specifically when used against viruses.

E1 demonstrated higher activity against SARS-CoV-2 at low doses. The E1 composition thus exhibited superior in-vivo anti-viral efficacy compared with CE3 consistent with the lower MIC recorded for the copolymer prepared at temperatures less than 15° C.

### Example 7—SARS-CoV-2—Human Airway Epithelial (HAE) Cells

Part A

COVID Organoid Protocol: Organoids comprising human airway epithelial (HAE) cells were infected by inoculation with SARS-CoV-2 virus and incubated at 37 deg C. with varying concentrations of CE3 (H:31 ppm, M:10 ppm, L:3 ppm) and E1 (H:82 ppm, M:41 ppm, L:8 ppm) and viral load measured by the number of PFUs (plaque-forming units of virus) assessed at time-points. The Control was polyethylene glycol (PEG) 200.

Data indicates concentration-dependent reductions from baseline of the SARS-CoV-2 (COVID-19) virus by CE3 and E1 as compared to a control group. The SARS-CoV-2 virus is the cause of the global COVID-19 pandemic. The concentrations utilized were far lower than the suite of pre-clinical data on the copolymer of CE3 intravenous infusion program.

The concentration dependent reduction in viral infection was determined differing concentrations of the two copolymer compositions and are shown in FIG. 6 of the attached drawings.

The data indicated a higher activity of composition E1 compared with CE3 against SARS-CoV-2 particularly at low doses.

Part B

In a separate study, CE3 and E1 indicated an excellent toxicity profile with less than 0.25% effect on Vero (monkey) cells at the concentrations tested.

Cytotoxicity Testing in Vero Cells Protocol: The cytotoxicity of CE3 (153 ppm, 76 ppm, 38 ppm, 19 ppm, 13 ppm, 6 ppm, 3 ppm, 2 ppm, 1 ppm) and E1 (82 ppm, 55 ppm, 41 ppm, 33 ppm, 27 ppm, 16 ppm, 12 ppm, 8 ppm, 4 ppm) across a range of concentrations was assessed in a Vero cell luminescence assay, and cell viability measured at time-points of 1 hour, 24 hours and 72 hours incubation, using a Control of untreated healthy Vero cells. At all time-points and concentrations measured, both compounds demonstrated minimal cytotoxic effects, with more than 99% of tested cells retaining their viability.

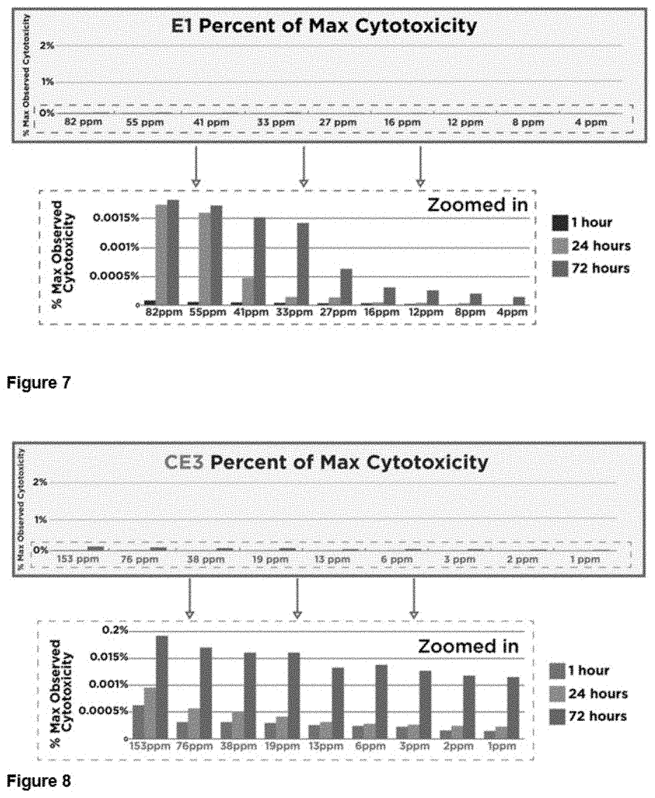

The results of the cytotoxicity testing is shown in FIG. 7 for E1 and FIG. 8 for CE3. For each concentration the percent of max cytotoxicity is reported (from left to right) for 1 hour, 24 hours and 72 hours.

### Example 8—Efficacy of E1 and CE3 Against Neisseria gonorrhoea (ATCC700825) in Mice

Summary

The aim of the study was to evaluate the efficacy of the compositions of Example E1 and Comparative Example CE3 against Neisseria gonorrhoea (ATCC700825) in the mouse vaginal infection model. Meropenem showed significant bactericidal activity in vaginal load, following an IV bolus dose of 50 mg/kg for seven days, when compared to the vehicle control (p<0.05). E1 showed dose dependent antibacterial effect. E1 at doses of 25 and 50 mg/kg (IV bolus for 7 days) showed mean dose dependent decrease in bacterial load, however they were not particularly significant; E1 at 100 mg/kg (IV bolus for 7 days) showed significant antibacterial effect, when compared to vehicle control (p<0.05).

CE3, showed significant dose dependent antibacterial effect in vaginal load at 100, 500 and 1000 mg/kg (IV bolus for 7 days), when compared to vehicle control seven days PI (p<0.05).

Test Items

CE3 and E1. were in aqueous solutions of 1000 mg/ml.

Test System Species: Mouse

Strain, Sex: BALB/c,
Female

Age: 4-6 weeks

Number of group: 9

Total number of animals: 90

N. gonorrhoeae Inoculation

Animals in the dioestrus stage of the oestrus cycle were implanted with a 5 mg, 21-day controlled-release oestradiol pellet subcutaneously and were treated with streptomycin and trimethoprim throughout the infection to increase their susceptibility to N. gonorrhoeae. Two days after pellet implantation, mice were inoculated intra-vaginally with N. gonorrhoeae (˜2×106 CFU/animal).

Formulation

The vehicle for CE3, E1 and Meropenem was sterilized 0.9% saline. The test compounds were diluted to the required concentrations with vehicle.

Treatment

Two days post-infection animals were treated for seven consecutive days as shown in the experimental design.

TABLE 14
Clinical signs in infected mice treated with test compounds.

| | Animal | |
|---|---|---|
| Group | No. | Clinical signs |
| Uninfected control | 1-10 | N |
| Vehicle control | 11-20 | N |
| Infected control + Meropenem | 21-30 | N |
| (50 mg/kg) | | |
| Infected E1 (25 mg/kg) | 31-40 | N |
| Infected + E1 (50 mg/kg) | 41-50 | N |
| Infected + E1 (100 mg/kg) | 51-60 | N |
| Infected + CE3 (100 mg/kg) | 61-70 | N |
| Infected + CE3 (500 mg/kg) | 71-80 | N |
| Infected + CE3 (1000 mg/kg) | 81-90 | N |

N: Apparently Normal

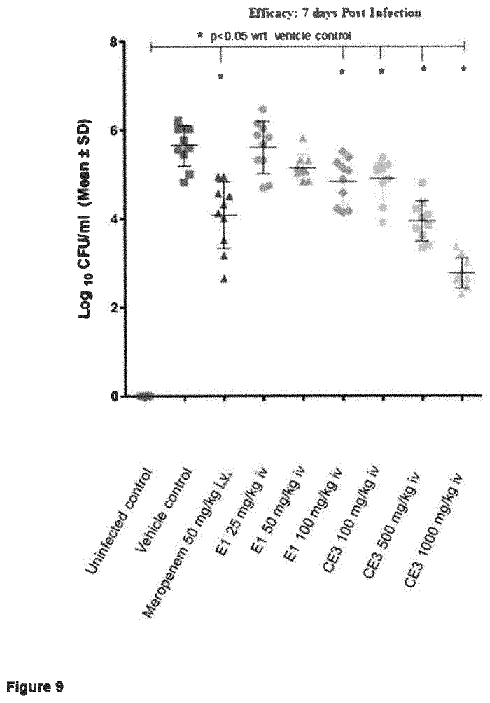

The results are depicted in FIG. 9 showing bacterial load in vaginal swabs following treatment with reference and test items in mice. *P<0.05 significantly different from vehicle control.

Conclusion

In the mouse vaginal infection model infected with N. gonorrhea (ATCC700825), Meropenem showed significant bactericidal activity in vaginal load, following a IV bolus dose of 50 mg/kg for seven days, when compared to the vehicle control (p<0.05).

E1 showed dose dependent antibacterial effect. E1 at doses of 25 and 50 mg/kg (IV bolus for 7 days) showed mean dose dependent decrease in bacterial load; E1 at 100 mg/kg (IV bolus for 7 days) showed significant antibacterial effect, when compared to vehicle control (p<0.05).

CE3, showed significant dose dependent antibacterial effect in vaginal load at 100, 500 and 1000 mg/kg (IV bolus for 7 days), when compared to vehicle control seven days PI (p<0.05).

The E1 composition thus exhibited superior in-vivo efficacy compared with CE3 consistent with the lower MIC recorded for the copolymer prepared at temperatures less than 15° C.

### Example 9—MIC Determination of Compositions of the Invention Prepared at 5° C. and 10° C.

The Compositions of the invention were prepared at 5° C. (Example 2—E2) and 10° C. (Example 1 E1) using the procedure of Example 1.

The MIC of S. aureus and E. coli was determined for the copolymer of each of Example 1 and Example 2 is reported in Table 15.

TABLE 15

| Example | MIC (ppm) S. aureus | MIC (ppm) E. coli |
|---|---|---|
| E1 (10° C.) | 20 | 30 |
| E2 (5° C.) | 20 | 20 |

1. A process for preparation of a biologically active polymer comprising an acrolein derived segment and a polyalkylene glycol oligomer, the process comprising reacting polyalkylene glycol with acrolein in aqueous solution to form a copolymer of molecular weight no more than 1000 Daltons at a temperature of no more than 15° C.

2. The process of claim 1, wherein the pH of the solution is alkaline and no more than pH 12.5.

3. The process of claim 1, wherein the aqueous solution of polyalkylene glycol and acrolein comprises water in an amount of at least 20% w/w.

4. The process of claim 1, wherein the weight ratio of polyalkylene glycol:acrolein is at least 4:1.

5. The process of claim 1, wherein the molecular weight of the polyalkylene glycol is no more than 800 Daltons.

6. The process of claim 1, wherein the molecular weight of the polyalkylene glycol is from 200 to 600 Daltons.

7. The process of claim 1, wherein the acrolein is added to an aqueous solution of polyalkylene glycol.

8. The process of claim 1, wherein the temperature is in the range of −20° C. to 15° C.

9. The process of claim 1, wherein the temperature of the aqueous solution is no more than 12° C.

10. The process of claim 1, wherein the temperature of the aqueous solution is at least −10° C.

11. The process of claim 1, wherein the temperature of the aqueous solution is −5° C. to 10° C.

12. The process of claim 1, wherein the acrolein is added to an aqueous solution of polyethylene glycol comprising at least 20% w/w water wherein the acrolein is added in the form of an aqueous solution of acrolein of concentration no more than 50% w/w, preferably no more than 30% w/w.

13. The process of claim 1, further comprising the following steps:
providing a mildly basic, preferably of pH no more than 12.5; more preferably of pH 8 to 12.5 such as 9.0 to 12.0, aqueous solution of a polyalkylene glycol, preferably polyethylene glycol of molecular weight in the range of from 200 to 600 Daltons;
adding, preferably slowly over a period such as at least 2 minutes, more preferably at least 5 minutes, acrolein as an aqueous solution of concentration no more than 50% w/w of the acrolein aqueous solution, optionally containing preservative;
maintaining the solution at a temperature of no more than 15° C., preferably no more than 12° C. during addition of the acrolein to form the copolymer; and
preferably once the acrolein monomer has been consumed, adding acid to provide a pH less than 9 and preferably no more than 8.

14. The process of claim 1, wherein the reaction of polyalkylene glycol with acrolein in aqueous solution is carried out in a reaction vessel comprising a heat exchanger for controlling the temperature.

15. The process of claim 14 wherein the reaction of polyalkylene glycol with acrolein in aqueous solution is conducted in a stirred reaction vessel provided with a jacket of flowing coolant liquid for controlling temperature within the reaction vessel.

16. The process of claim 14, wherein the reaction conditions are regulated by computer control of one or more reaction parameters selected from rate of acrolein addition, rate of flow of water, temperature of water within the jacket and speed at which the aqueous solution is stirred.

17. A method of treatment of a subject suffering a disease selected from microbial infection, viral infection and cancer comprising administering to the subject an effective amount of the biologically active polymer prepared according to the process of claim 1.

18. The method of claim 17, wherein the disease is a parenteral disease.

19. A composition for treatment of a disease in a subject selected from bacterial infection, viral infection and cancer wherein said composition is prepared by a process comprising the process of claim 1.
